(19) United States
(12) Patent Application Publication
(10) Pub. No.: US 2012/0264952 A1
(43) Pub. Date: Oct. 18, 2012

(54) CATALYST FOR PREPARING ETHYLENE OXIDE

(75) Inventors: Tobias Rosendahl, Mannheim (DE); Torsten Mäurer, Ludwigshafen (DE); Cornelia Katharina Dobner, Ludwigshafen (DE); Jürgen Zühlke, Speyer (DE)

(73) Assignee: BASF SE, Ludwigshafen (DE)

(21) Appl. No.: 13/446,405

(22) Filed: Apr. 13, 2012

Related U.S. Application Data
(60) Provisional application No. 61/475,259, filed on Apr. 14, 2011.

(51) Int. Cl. B01J 23/50; C01F 7/02; C07D 301/03; B01J 21/04; B01J 23/68

(52) U.S. Cl. 549/534; 502/347; 502/348; 502/317; 502/216; 502/324; 423/625

(57) ABSTRACT

Catalyst for preparing ethylene oxide, at least comprising silver applied to a support, the support having a BET constant C in the range from 0 to 800, determined in accordance with DIN ISO 9277.

The present invention relates to a catalyst for preparing ethylene oxide, at least comprising silver applied to a support, the support having a BET constant C in the range from 0 to 800, and the constant being determined in accordance with DIN ISO 9277. The present invention further relates to this catalyst support per se. The present invention additionally relates to a process for preparing the catalyst, to the catalyst obtainable by this process, and to the use of the catalyst for oxidizing ethylene to ethylene oxide. The present invention additionally relates to a process for preparing ethylene oxide from ethylene, comprising oxidizing ethylene in the presence of the stated catalyst.

Ethylene oxide is an important basic chemical and industrially is frequently prepared by direct oxidation of ethylene with oxygen in the presence of catalysts comprising silver. Oftentimes support catalysts are used, to which the catalytically active, metallic silver has been applied by means of a suitable process. Support materials used may in principle be various porous materials, such as, for example, activated carbon, titania, zirconia or silica, or ceramic masses, or mixtures of these materials. Generally speaking, alpha-alumina is used as a support. For examples of the direct oxidation of ethylene, reference may be made to DE-A-2300512, DE-A 2521906, EP-A-0014457, DE-A-2454972, EP-A-0172565, EP-A-0357293, EP-A-0266015, EP-A-0011356, EP-A-0085237, DE 2560684 or DE-A-2753359.

Besides silver as the active component, these catalysts often include promoters for improving the catalytic properties. Examples include alkali metal compounds and/or alkaline earth metal compounds as promoters. Some documents teach the use of transition metals such as tungsten or molybdenum. One particularly preferred promoter for influencing the activity and selectivity of catalysts is rhenium. Catalysts which comprise rhenium and/or other transition metal promoters in combination with alkali metal compounds and/or alkaline earth metal compounds are used preferentially in industry because of their high selectivity. By selectivity is meant the molar percentage of ethylene that reacts to form ethylene oxide. The activity is characterized by the concentration of ethylene oxide in the reactor product under otherwise constant conditions such as temperature, pressure, gas quantity, catalyst quantity, etc., for example. The higher the ethylene oxide concentration in the reactor product stream, the higher the activity of the catalyst. The lower the temperature needed in order to attain a defined concentration of alkylene oxide, the higher the activity. In order to achieve a high selectivity, the combination of the active metal with the promoters, and the composition of the support, are typically harmonized with one another in order to give catalysts having the best possible properties.

Starting out from this prior art, one object of the present invention was to provide new catalysts for the epoxidation of ethylene, having advantageous activities and/or selectivities.

A further object of the present invention was to provide suitable supports for the preparation of catalysts having particularly advantageous profiles of properties for the epoxidation of ethylene.

In accordance with the invention, this object is achieved by means of a catalyst for preparing ethylene oxide, comprising silver, applied to a support, the support having a BET constant C in the range from 0 to 800, determined in accordance with DIN ISO 9277. Surprisingly it has been found that the use of supports having a BET constant C within a defined range leads to particularly advantageous properties on the part of the resultant catalyst. The catalysts, more particularly, exhibit advantageous selectivities in the preparation of ethylene oxide.

The invention accordingly provides a catalyst for preparing ethylene oxide, comprising silver, applied to a support, the support having a BET constant C in the range from 0 to 800, determined in accordance with DIN ISO 9277.

The invention further provides the support per se, in other words a catalyst support for a catalyst for preparing ethylene oxide, the support having a BET constant C in the range from 0 to 800, determined in accordance with DIN ISO 9277.

The invention further provides a process for preparing a catalyst for preparing ethylene oxide, and also the catalyst obtainable or obtained by this process, the process comprising the steps of
(i) providing a support having a BET constant C in the range from 0 to 800, determined in accordance with DIN ISO 9277, and
(ii) applying silver to the support from (i).

The invention further provides for the use of the catalyst of the invention, or the catalyst obtainable by the process of the invention, for epoxidizing ethylene.

With regard to the preparation of the catalyst, generally all processes are suitable in which silver is applied to the support in a suitable way. It is preferred here for at least one mixture comprising silver to be applied by means, for example, of impregnating or spraying or mixing methods to the support, as described in more detail further on below.

With regard to the support, it is characterized by a BET constant C in the range from 0 to 800, preferably in the range from 0 to 700, more preferably in the range from 0 to 600, more preferably in the range from 25 to 500, and very preferably in the range from 50 to 450. The BET constant C (also the C value) here is determined in accordance with DIN ISO 9277 (May 2003).

The determination of the C value is based on the method of Brunnauer, Emmet, and Teller for determining the total specific surface area of porous solids by measurement of the physisorbed gas quantity (BET), as described in Journal of the American Chemical Society, Vol. 60, pp. 301-319 (1938). Drawing on both a gas kinetics model and a statistical model, Brunnauer, Emmet, and Teller, for the physisorption of gases, derived a simple relation between gas pressure and gas quantity absorbed, referred to as the BET isotherm. The relation is conveyed by the formula

$$V = \frac{{V}_{M} \cdot C \cdot {p}_{r}}{( 1 - {p}_{r} ) \cdot ( 1 - {p}_{r} + C \cdot {p}_{r} )}$$

where V is the molar volume of the adsorptive, i.e. of the gaseous, liquid or dissolved substance to be adsorbed. V_{M} is the gas volume needed in order to form a monolayer; pᵣ=p/p₀, i.e., the relative pressure, with p being the equilibrium pressure and p₀ the saturation vapor pressure of the gas at the measurement temperature. C is the BET constant, and is subject to the following relationship:

C=A·exp[(E₁−E_{L})/RT]

where E₁ is the adsorption energy on formation of the first layer, E_{L} is the adsorption energy of the second and higher layers, A is the kinetic factor, and R is the gas constant. T stands for the respective temperature in kelvins.

The C value constitutes a statement concerning the interaction energy of the adsorbate with the support, and provides information on the polarity of the support surface. Small C values are characteristic of nonpolar surfaces, whereas large C values are an indicator of highly polar or microporous surfaces. The C value is dependent, for example, on the composition of the support, the acidity of the surface, and the porosity, and also on the form of the pores. Thus, for example, the calcining conditions used in preparing the support, or the addition of polar compounds (additions of silicon) or of nonpolar compounds (e.g., waxes) to the support material, have an influence on the C value.

Supports suitable in accordance with the invention can be prepared by processes known from the prior art. Reference may be made, for example, to US 2009/0198076 A1, WO 2006/133187, WO 03/072244, US 2005/0096219 A1, or EP 0 496 386 B2.

Examples of suitable inert support materials are alumina, silica, silicon carbide, titania, zirconia, and mixtures thereof, with alumina being preferred. In one preferred embodiment, accordingly, the present invention provides a catalyst as described above, and a support as described above, where the support is an alumina support. Furthermore, the present invention also provides a catalyst as described above which is obtainable by the process described above, the support being an alumina support.

The term alumina as presently used encompasses all conceivable structures such as alpha-, gamma- or tetha-alumina. In one preferred embodiment, the support is an alpha-alumina support. The present invention also, accordingly, provides a process as described above and a catalyst and a support obtainable by the process, the support being an alpha-alumina. The present invention additionally provides, as well, a catalyst as described above, per se, the support being an alpha-alumina support.

In a further-preferred embodiment, the alpha-alumina has a purity of at least 75%, preferably a purity of at least 80%, more preferably a purity of at least 85%, more preferably a purity of at least 90%, more preferably a purity of at least 98%, more preferably a purity of at least 98.5%, and very preferably a purity of at least 99%.

The term alpha-alumina, accordingly, also encompasses alpha-aluminas which comprise constituents additionally, examples being elements selected from the group consisting of zirconium, alkali metals, alkaline earth metals, silicon, zinc, gallium, hafnium, boron, fluorine, copper, nickel, manganese, iron, cerium, titanium, chromium, and mixtures of two or more of these elements.

Generally speaking, a suitable catalyst support for the present invention can be prepared by mixing the alumina with water or another suitable liquid with a burnout material or a pore former and at least one binder. Examples of suitable pore formers include cellulose and derivatives of cellulose, such as methylcellulose, ethylcellulose, carboxymethylcellulose, for example, or polyolefins, such as polyethylenes and polypropylenes, or natural burnout materials such as finely ground walnut shells, for example. The pore formers are selected so that they are burnt out fully at the selected oven temperatures of the calcination of the alumina to form the finished alpha-alumina support. Suitable binders and/or extrusion auxiliaries are described in EP 0 496 386 B2, for example. Examples include alumina gels with nitric acid or acetic acid, cellulose, e.g., methylcellulose, ethylcellulose, or carboxyethylcellulose, or methyl or ethyl stearate, polyolefin oxides, waxes, and the like.

The paste formed by mixing can be brought to the desired shape by extrusion. Extrusion auxiliaries may be used to support the extrusion procedure.

After having been shaped, the shaped body obtained as described above is typically, optionally, dried and calcined to give the alumina support according to (i). Calcining takes place typically at temperatures in the range from 1200° C. to 1600° C. It is common to wash the alumina support after calcining, in order to remove soluble constituents.

The alpha-alumina here may comprise the constituents in any suitable form—for example, as an element and/or in the form of one or more compounds. Where the alpha-alumina here comprises one or more constituents in the form of a compound, it comprises this in the form, for example, of oxide or mixed oxide. The present invention thus also describes an alpha-alumina comprising at least one further constituent selected from the group consisting of silica, sodium oxide, potassium oxide, calcium oxide, and magnesium oxide, nickel oxide, gallium oxide, hafnium oxide, copper oxide, iron oxide, and mixed oxides thereof.

With regard to the amount of the further constituents, the total amount of the further constituents is preferably situated in a range of less than 25% by weight, more preferably less than 20% by weight, more preferably less than 15% by weight, more preferably less than 10% by weight, more preferably less than 5% by weight, more preferably less than 2% by weight, more preferably less than 1.5% by weight, and very preferably less than 1% by weight, based on the total weight of the support.

Where the support comprises, for example, silicon, it comprises it preferably in an amount in the range from 50 to 10000 ppm, more preferably in an amount of 50 to 5000 ppm, more preferably in an amount of 50 to 800 ppm, based on the total weight of the support and calculated as the element.

Where the support comprises, for example, alkali metals, it comprises them preferably in a total amount in the range from 10 to 2500 ppm, more preferably in an amount of 10 to 1000 ppm, more preferably in an amount of 50 to 850 ppm, based on the total weight of the support and calculated as the element. In one embodiment the support comprises at least one alkali metal selected from the group consisting of sodium and potassium. Where the support comprises sodium, for example, it comprises it preferably in an amount in the range from 10 to 1500 ppm, more preferably in an amount of 10 to 800 ppm, more preferably in an amount of 10 to 500 ppm, based on the total weight of the support and calculated as the element. Where the support comprises potassium, for example, it comprises it preferably in an amount in the range from 10 to 1000 ppm, more preferably in an amount of 10 to 500 ppm, more preferably in an amount of 10 to 300 ppm, based on the total weight of the support and calculated as the element. In one embodiment of the invention the support comprises, for example, sodium in an amount of 10 to 1500 ppm and potassium in an amount of 10 to 1000 ppm.

The present invention also describes, accordingly, a process as described above for preparing a catalyst, and a catalyst obtainable by this process, where the support comprises sodium in an amount of 10 to 1500 ppm, potassium in an amount of 10 to 1000 ppm, and cesium in an amount of 0 to 1000 ppm, the support more preferably comprising sodium in an amount of 10 to 500 ppm, potassium in an amount of 10 to 300 ppm, and cesium in an amount of 0 to 100 ppm, based on the total weight of the support and calculated as the elements. The invention additionally describes a catalyst obtainable by this process, and also the support per se. The present invention also provides, moreover, a catalyst as described above, and also a support per se as described above, the support comprising sodium in an amount of 10 to 1500 ppm; potassium in an amount of 10 to 1000 ppm, and cesium in an amount of 0 to 100 ppm, more preferably sodium in an amount of 10 to 500 ppm, potassium in an amount of 10 to 300 ppm, and cesium in an amount of 0 to 100 ppm, based in each case on the total weight of the support and calculated as the elements.

Where the support comprises, for example, alkaline earth metals, it comprises them preferably in a total amount in the region of not more than 2500 ppm, as for example in the range from 10 to 2500 ppm, more preferably in an amount of 10 to 1200 ppm, more preferably in an amount of 10 to 700 ppm, based on the total weight of the support and calculated as the element. In one embodiment the support comprises at least one alkaline earth metal selected from the group consisting of calcium and magnesium. Where the support comprises calcium, for example, it comprises it preferably in an amount in the range from 10 to 1500 ppm, more preferably in an amount of 10 to 1000 ppm, more preferably in an amount of 10 to 500 ppm, based on the total weight of the support and calculated as the element. Where the support comprises, for example, magnesium, it comprises it preferably in an amount in the range from 10 to 800 ppm, more preferably in an amount of 10 to 500 ppm, more preferably in an amount of 10 to 250 ppm, based on the total weight of the support and calculated as the element.

The present invention also describes, accordingly, a process as described above for preparing a catalyst, and a catalyst obtainable by this process, where the support comprises magnesium in an amount of 10 to 800 ppm, and calcium in an amount of 10 to 1500 ppm, in each case based on the total weight of the support and calculated as the elements. The present invention also, moreover, provides a catalyst as described above, and also a support per se as described above, the support comprising magnesium in an amount of 10 to 800 ppm, and calcium in an amount of 10 to 1500 ppm, based in each case on the total weight of the support and calculated as the elements. With particular preference the support comprises, for example, sodium in an amount of 10 to 1500 ppm, potassium in an amount of 10 to 1000 ppm, magnesium in an amount of 10 to 800 ppm, and calcium in an amount of 10 to 1500 ppm, in each case based on the total weight of the support and calculated as the elements.

Where the support comprises, for example, silicon, it comprises it preferably in an amount in the range from 50 to 10000 ppm, more preferably in an amount of 50 to 5000 ppm, more preferably in an amount of 50 to 600 ppm, based on the total weight of the support and calculated as the element.

One support preferred in accordance with the present invention is, for example, an alpha-alumina in a purity of at least 90%, comprising 50 to 10000 ppm of silicon, from 10 to 1500 ppm of sodium, and from 10 to 2500 ppm of alkaline earth metals in total, in each case calculated as the elements and based on the total weight of the support. The support preferably comprises calcium and/or magnesium as alkaline earth metal. Particular preference is given to an alpha-alumina in a purity of at least 98% by weight that comprises 50 to 5000 ppm of silicon, 10 to 800 ppm of sodium, and 10 to 700 ppm of alkaline earth metals in total, in each case calculated as the element and based on the total weight of the support.

The supports used in accordance with the invention preferably have a BET surface area, determined in accordance with DIN ISO 9277, of 0.1 to 5 m²/g, more preferably in the range from 0.1 to 2 m²/g, more preferably in the range from 0.5 to 1.5 m²/g, more preferably in the range from 0.6 to 1.3 m²/g, and very preferably in the range from 0.6 to 1.0 m²/g, determined in accordance with DIN ISO 9277.

Further, the supports of the invention preferably have pores having diameters in the range from 0.1 to 100 μm, it being possible for the pore distribution to be monomodal or polymodal—for example, bimodal, trimodal or tetramodal. The supports preferably have a bimodal pore distribution. With further preference the supports have a bimodal pore distribution having peak maxima in the range from 0.1 to 10 μm and 15 to 100 μm, preferably in the range from 0.1 to 5 μm and 17 to 80 μm, more preferably in the range from 0.1 to 3 μm and 20 to 50 μm, more preferably in the range from 0.1 to 1.5 μm and 20 to 40 μm. The pore diameters are determined by Hg porosimetry (DIN 66133). The term “bimodal pore distribution having peak maxima in the range from 0.1 to 10 μm and 15 to 100 μm”, as used above, is saying that one of the two peak maxima is situated in the range from 0.1 to 10 μm and the other peak maximum is situated in the range from 15 to 100 μm.

The present invention also describes, accordingly, a process for preparing a catalyst, as described above, and a catalyst obtainable by this process, the support having a bimodal pore distribution, preferably a bimodal pore distribution at least comprising pores having a pore diameter in the range from 0.1 to 15 μm and pores having a pore diameter in the range from 15 to 100 μm, determined by Hg porosimetry. The present invention also provides, moreover, a catalyst as described above, and also a support per se as described above, the support having a bimodal pore distribution, preferably a bimodal pore distribution at least comprising pores having a pore diameter in the range from 0.1 to 10 μm and pores having a pore diameter in the range from 15 to 100 μm, determined by Hg porosimetry (DIN 66133).

The geometrical shape of the supports is generally of minor importance; usefully, however, the supports ought to take the form of particles which allow unhindered diffusion of the reaction gases to as large a proportion as possible of the exterior and interior surface area of the support, coated with the catalytically active, with silver particles and coated optionally with further promoters. Furthermore, the geometrical shape selected for the support must ensure a minimal loss of pressure over the total reactor length. In one preferred embodiment, the support is used in the form of a shaped body—for example, as an extrudate, hollow extrudate, star extrudate, bead, ring or hollow ring. The support is preferably a shaped body having the geometry of a hollow body. Especially preferred are cylinders having the following geometries (outer diameter×length×inner diameter, indicated in mm in each case): 5×5×2, 6×6×3, 7×7×3, 8×8×3, 8×8.5×3, 8×8.5×3.5, 8.5×8×3.5, 8.5×8×3, 9×9×3, 9.5×9×3, 9.5×9×3.5. Each length figure encompasses tolerances in the region of +/−0.5 mm.

It is also possible in accordance with the invention for the catalyst to be used in the form of a crushed catalyst material obtained from one or more of the stated shaped bodies.

The water absorption of the supports is situated, for example, in the range from 0.35 ml/g to 0.65 ml/g, preferably in the range from 0.42 ml/g to 0.52 ml/g, determined by vacuum cold-water absorption.

The catalyst of the invention comprises silver as active metal. The catalyst here may comprise silver in an amount of, for example, 5% to 35% by weight, more particularly of 10% to 30% by weight, preferably in an amount of 10% to 25% by weight, based on the total weight of the catalyst and calculated as the element.

The present invention also describes, accordingly, a process as described above, and a catalyst obtainable by the process, and also a catalyst per se as described above, comprising silver in an amount of 5% to 35% by weight, based on the total weight of the catalyst.

In one preferred embodiment, the catalyst, besides silver, further comprises at least one promoter—for example, six, five, four, three or two promoters or one promoter. A promoter for the purposes of the invention is a constituent of the catalyst through which, in comparison to a catalyst not containing the constituent, an improvement is achieved in one or more catalytic properties, such as selectivity, activity, conversion and/or yield or space-time yield. Preferred compounds are those which are chemically largely stable under the reaction conditions and do not catalyze unwanted reactions.

With regard to the at least one promoter, all promoters known in the prior art are conceivable. The at least one promoter is preferably selected from the group consisting of lithium, sodium, potassium, rubidium, cesium, beryllium, magnesium, calcium, strontium, barium, manganese, rhenium, cadmium, tungsten, molybdenum, chromium, sulfur, and mixtures of two or more thereof. With particular preference the catalyst comprises at least one promoter selected from the group consisting of rhenium, cesium, lithium, tungsten, chromium, manganese, sulfur, and mixtures of two or more thereof. The catalyst more preferably comprises at least rhenium as promoter, and at least one further promoter selected from the group consisting of cesium, lithium, tungsten, chromium, manganese, sulfur, and mixtures of two or more thereof.

The present invention also describes, accordingly, a process as described above, and a catalyst obtainable by the process, and also a catalyst per se as described above, comprising at least rhenium as promoter, preferably comprising silver in an amount of 5% to 35% by weight, based on the total weight of the catalyst, and at least rhenium as promoter.

Where the catalyst, as described above, comprises rhenium, it comprises rhenium preferably in an amount of 50 to 600 ppm, more preferably in an amount of 100 to 450 ppm, more preferably in an amount of 150 to 400 ppm, based on the total weight of the catalyst and calculated as the element.

In one particularly preferred embodiment, the catalyst further comprises at least one further promoter selected from the group consisting of lithium, sodium, potassium, rubidium, cesium, beryllium, magnesium, calcium, strontium, barium, manganese, rhenium, cadmium, tungsten, chromium, molybdenum, sulfur, and mixtures of two or more thereof. More preferably the catalyst further comprises at least one promoter selected from the group consisting of cesium, lithium, tungsten, chromium, manganese, sulfur, and mixtures of two or more thereof, and mixtures of two or more thereof. In one particularly preferred embodiment, the catalyst at least further comprises cesium, lithium, tungsten, and sulfur; more particularly the catalyst comprises rhenium and, further, cesium, lithium, tungsten, and sulfur, as promoters.

Where the catalyst comprises at least one further promoter, it preferably comprises a total amount of these promoters in an amount of 10 to 2000 ppm, preferably in each case in an amount of 10 to 1500 ppm, more preferably in each case in an amount of 50 to 1300 ppm, and with particular preference in each case in an amount of 80 to 1300 ppm, based on the total weight of the catalyst and calculated as the sum of the elements.

Where the catalyst comprises tungsten, for example, it comprises it preferably in an amount of 10 to 500 ppm, preferably in an amount of 50 to 300 ppm.

Where the catalyst comprises cesium, for example, it comprises it preferably in an amount of 20 to 850 ppm, more particularly in an amount of 100 to 600 ppm, based on the total weight of the catalyst and calculated as the element.

Where the catalyst comprises lithium, for example, it comprises it preferably in an amount of 10 to 450 ppm, more particularly in an amount of 50 to 300 ppm, based on the total weight of the catalyst and calculated as the element.

Where the catalyst comprises sulfur, for example, it comprises it preferably in an amount of 5 to 300 ppm, more particularly in an amount of 5 to 150 ppm, based on the total weight of the catalyst and calculated as the element.

In one particularly preferred embodiment, the catalyst comprises rhenium in an amount of 150 to 450 ppm, tungsten in an amount of 50 to 300 ppm, cesium in an amount of 100 to 600 ppm, lithium in an amount of 50 to 300 ppm, and sulfur in an amount of 5 to 150 ppm.

With regard to the application of silver, it may be applied to the support by all of the impregnating and deposition methods of the prior art for preparing silver catalysts for preparing ethylene, oxide, it being possible for these methods to comprise one or more impregnating and calcining stages. Reference may be made, by way of example, to the silver catalyst preparation processes as disclosed in DE-A 2300512, DE-A 2521906, EP-A 0 014 457, EP-A 0 085 237, EP-A 0 0384 312, DE-A 2454972, DE-A 3321895, EP-A 0 229 465, DE-A 3150205, EP-A 0 172 565, and EP-A 0 357 293.

Where the catalyst, in addition to silver, comprises at least one promoter, it is preferred to apply to the support at least one mixture comprising silver and at least one promoter, by means, for example, of impregnating or spraying or mixing methods. The sequence of application of the at least one promoter and of silver in this case is generally arbitrary; in other words, embodiments are encompassed in which silver and the at least one promoter are applied at the same time to the support. Also encompassed are embodiments in which silver and the at least one promoter are applied in different steps to the support, the sequence of the steps being generally arbitrary. Additionally encompassed are embodiments in which a portion of the at least one promoter, which is applied to the support only following application of the silver, and the remaining portion is applied simultaneously with silver. It is preferred to apply silver and the at least one promoter simultaneously to the support.

The silver is applied preferably in the form of a silver compound, which may be a salt or a silver complex, to the support of the invention. It is preferred to apply the silver compound in solution, more particularly in solution in water. In order to obtain the silver compound in soluble form, the silver compound, such as silver(I) oxide or silver(I) oxalate, for example, may also be suitably admixed with complexing agent, such as ethanolamine, EDTA, 1,3- or 1,2-propanediamine, ethylenediamine and/or alkali metal oxalate, which may also act as a reducing agent at the same time. It is particularly preferred to apply silver in the form of a silver amine compound, more preferably a silver ethylenediamine compound.

The present invention also provides, accordingly, a process as described above in which in step (ii) a mixture comprising silver in the form of a silver amine compound, preferably in the form of silver ethylene-diamine compound, is applied to the support described above. The present invention likewise provides a catalyst obtainable by this process.

The application of the silver may be followed afterwards by at least one aftertreatment step, an example being a drying step, e.g., one, two or more drying steps. Drying in this case takes place typically at temperatures in the range from 10 to 200° C. The aftertreatment step preferably comprises a reduced-pressure treatment. Accordingly, in one preferred embodiment, the support is evacuated following application. The evacuating takes place preferably at a pressure in the region of not more than 500 mbar, more preferably at a pressure of not more than 250 mbar, and very preferably at a pressure of not more than 30 mbar, and also, preferably, at a temperature in the range from 2° C. to 50° C., more preferably at a temperature in the range from 5° C. to 30° C., and very preferably at room temperature. The reduced-pressure treatment takes place here, for example, for a time of at least 1 min, preferably of at least 5 min, more preferably for a time in the range from 5 min to 120 min, more particularly in the range from 10 min to 45 min, with particular preference in the range from 10 min to 20 min. Application of the silver and, optionally, the at least one drying step are followed preferably by at least one calcining step.

The present invention accordingly also describes a process for preparing a catalyst, as described above, and also the catalyst obtainable by this process, further comprising a step (iii) of
(iii) calcining the optionally aftertreated support obtained from (ii).

The calcining takes place at temperatures, for example, in a range from 150 to 750° C., generally in the range from 200 to 500° C., preferably in the range from 220 to 500° C., and more preferably in the range from 250 to 350° C., the calcining time being generally at least 5 minutes or more, as for example in the range from 5 minutes to 24 h or in the range from 10 minutes to 12 h. With particular preference the calcining time is situated in the range from 5 minutes to 3 hours. The calcining here may take place at a constant temperature; additionally encompassed are embodiments in which the temperature is altered continuously or discontinuously during the calcining time. The calcining may take place under any gas atmosphere suitable for this purpose, with preference being given to air, nitrogen and/or lean air. In addition, the calcining is carried out preferably in a muffle furnace, in a rotary furnace, in a convection oven and/or in a belt calcination furnace.

Where the catalyst comprises at least one promoter, said at least one promoter, in the process of the invention for preparing the catalyst, is applied to the support preferably in the form of compounds, as for example in the form of complexes or in the form of salts, as for example in the form of halides, as for example in the form of fluorides, bromides or chlorides, or in the form of carboxylates, nitrates, sulfates or sulfides, phosphates, cyanides, hydroxides, carbonates, or as salts of heteropolyacids, in the form of salts, as for example in the form of salts of the heteropolyacids of rhenium and/or of tungsten.

Where the catalyst comprises rhenium, for example, the latter is applied preferably as a compound, in the form of, for example, a halide, oxyhalide, oxide, or as an acid. Moreover, rhenium may be employed, for example, as rhenate or perrhenate in the preparation process of the invention. If rhenium is to be used as a promoter, it is employed preferably in the form of a compound selected from the group consisting of ammonium perrhenate, rhenium(III) chloride, rhenium(V) chloride, rhenium(V) fluoride, rhenium(VI) oxide, and rhenium(VII) oxide. For the purposes of the invention, rhenium is applied in the form of ammonium perrhenate to the support, with particular preference.

Where the catalyst comprises tungsten as a promoter, as described above, the tungsten is applied preferably as a tungsten compound to the support. Here it is possible in principle to use any suitable tungsten compound. For example, tungsten is applied in the form of tungstate or tungstic acid.

Where the catalyst comprises lithium as a promoter, as described above, the lithium is applied preferably as a lithium compound to the support. Here it is possible in principle to use any suitable lithium compound. Lithium is applied preferably in the form of lithium nitrate.

Where the catalyst comprises cesium as a promoter, as described above, the cesium is applied preferably as a cesium compound to the support. Here it is possible in principle to use any suitable cesium compound. Cesium is applied preferably in the form of cesium hydroxide.

Where the catalyst comprises sulfur as a promoter, as described above, the sulfur is applied preferably as a sulfur compound to the support. Here it is possible in principle to use any suitable sulfur compound. It is preferred to apply sulfur in the form of ammonium sulfate.

The at least one promoter, more preferably the at least one promoter compound, is preferably dissolved in a suitable solution, preferably in water, before being applied. The support is then preferably impregnated with the solution obtained, comprising one or more of the promoters. Where two or more promoters are to be added, they may be applied to the support either together or separately in a single impregnating step or in two or more impregnating steps. With regard to the solution comprising one or more of the promoters, it may be prepared in any suitable way. For example, the promoters may each be dissolved separately in one solution each, and the resulting solutions, comprising one promoter in each case, may be used subsequently for the impregnation. It is likewise possible for two or more of the promoters to be dissolved together in a solution, and for the solution obtained then to be used for impregnation. It is possible, furthermore, for the solutions obtained that comprise at least one promoter to be combined prior to impregnation and for the resulting solution, comprising all of the promoters, to be applied to the support.

Where, for example, at least cesium, tungsten, lithium, sulfur, and rhenium are used as promoters, then, in accordance with one particularly preferred embodiment, at least one solution comprising cesium and tungsten, one further solution comprising lithium and sulfur, and one further solution comprising rhenium are prepared. The solutions are either applied to the support in separate impregnating steps, or are combined to form a solution before being applied and only subsequently used for impregnation. The solutions are applied to the support preferably together, more preferably together with the mixture comprising silver as silver amine compound, preferably as silver ethylenediamine compound.

Application may take place in principle by any suitable method, as for example by impregnation of the support. Application takes place with particular preference by vacuum impregnation at room temperature. In the case of vacuum impregnation, the support is treated preferably to start with at a pressure in the region of not more than 500 mbar, more preferably at a pressure of not more than 250 mbar, and very preferably at a pressure of not more than 30 mbar, and also, preferably, at a temperature in the range from 2° C. to 50° C., more preferably at a temperature in the range from 5° C. to 30° C., and more preferably at room temperature. The vacuum treatment takes place here, for example, for a time of at least 1 min, preferably of at least 5 min, more preferably for a time in the range from 5 min to 120 min, more particularly in the range from 10 min to 45 min, very preferably in the range from 15 min to 30 min. Following the vacuum treatment, the at least one solution, for example the mixture comprising silver, or at least one solution comprising at least one promoter, preferably the mixture comprising silver and the at least one promoter, is applied to the support. The solution is applied preferably dropwise or by spraying, preferably by spraying. Application in these cases takes place preferably by means of a nozzle. Following application, the support is preferably evacuated further. Evacuation takes place preferably at a pressure in the region of not more than 500 mbar, more preferably at a pressure of not more than 250 mbar, and very preferably at a pressure of not more than 30 mbar, and also, preferably, at a temperature in the range from 2° C. to 50° C., more preferably at a temperature in the range from 5° C. to 30° C., and very preferably at room temperature. The vacuum treatment takes place here, for example, for a time of at least 1 min, preferably of at least 5 min, more preferably for a time in the range from 5 min to 120 min, more particularly in the range from 10 min to 45 min, very preferably in the range from 10 min to 20 min.

With regard to the time at which the promoter is applied, this may be carried out following the above-described calcining of step (iii). An alternative possibility is to apply the at least one promoter together with the silver compound to the support. The invention, accordingly, encompasses embodiments in which the at least one promoter, i.e., for example, five different promoters, four different promoters, three different promoters, two different promoters or one promoter, are applied to the support and the support thus treated only then is calcined as described above to give a catalyst of the invention.

The present invention also describes, accordingly, a process for preparing a catalyst, and also the catalyst obtainable by this process, comprising the steps of (i) providing a support having a BET constant C in the range from 0 to 800, determined in accordance with DIN ISO 9277, (ii) applying silver and at least one promoter to the support by applying a solution comprising silver and the at least one promoter, and (iii) calcining the optionally dried support from (ii).

The promoters are preferably applied together with silver in step (ii).

The catalysts of the invention and the catalysts obtainable by the process of the invention are suitable especially as catalysts for preparing ethylene oxide from ethylene, comprising oxidizing ethylene. High selectivities and good activities are achieved.

In accordance with one further aspect, therefore, the present invention also provides a process for preparing ethylene oxide from ethylene, comprising oxidizing ethylene in the presence of a catalyst for preparing ethylene oxide, comprising silver, applied to a support, the support having a BET constant C in the range from 0 to 800, determined in accordance with DIN ISO 9277, or of a catalyst obtainable by a process for preparing a catalyst for preparing ethylene oxide as described above.

In a further embodiment, therefore, the present invention also provides the process for preparing ethylene oxide from ethylene, comprising oxidizing ethylene as described above, wherein the catalyst further comprises rhenium.

The present invention also provides, moreover, the use of a catalyst for preparing ethylene oxide, at least comprising silver, applied to a support, the support having a BET constant C in the range from 0 to 800, determined in accordance with DIN ISO 9277.

In accordance with the invention, the epoxidation may take place by any of the processes known to the skilled person. All of the reactors can be used that can be used in the prior-art ethylene preparation processes, examples being externally cooled shell-and-tube reactors (cf. Ullmann's Encyclopedia of Industrial Chemistry, 5^{th} edition, vol. A-10, pp. 117-135, 123-125, VCH-Verlagsgesellschaft, Weinheim 1987), or reactors with a loose catalyst filling and with cooling tubes, examples being the reactors described in DE-A 3414717, EP 0082609, and EP-A 0339748. The epoxidation takes place preferably in at least one tube reactor, preferably in a shell-and-tube reactor. The catalyst of the invention may be used either alone or in a mixture with other catalysts in a combined and/or structured catalyst bed.

For the preparation of ethylene oxide from ethylene and oxygen it is possible in accordance with the invention to operate under conventional reaction conditions, as they are described in, for example, DE 25 21 906 A1, EP 0 014 457 A2, DE 2 300 512 A1, EP 0 172 565 A2, DE 24 54 972 A1, EP 0 357 293 A1, EP 0 266 015 A1, EP 0 085 237 A1, EP 0 082 609 A1, and EP 0 339 748 A2. In this case it is possible additionally, to mix inert gases as well, such as nitrogen, or gases which behave inertly under the reaction conditions, such as steam, methane, and also, optionally, reaction moderators, examples being halides, hydrocarbons such as ethyl chloride, vinyl chloride or 1,2-dichloroethane into the reaction gas comprising ethylene and molecular oxygen. The oxygen content of the reaction gas is usefully in a range in which there are no explosive gas mixtures. One suitable composition of the reaction gas for preparing ethylene oxide may comprise, for example, an amount of ethylene in the range from 10% to 80% by volume, preferably from 20% to 60% by volume, more preferably from 25% to 50% by volume, and very preferably in the range from 30% to 40% by volume, based on the total volume of the reaction gas. The oxygen content of the reaction gas in this case is usefully within a region of not more than 10% by volume, preferably of not more than 9% by volume, more preferably of not more than 8% by volume, and very preferably of not more than 7% by volume, based on the total volume of the reaction gas.

The reaction gas preferably comprises a chlorine-containing reaction moderator such as ethyl chloride, vinyl chloride or dichloroethane in an amount of 0 to 15 ppm, preferably in an amount of 0.1 to 8 ppm. The remainder of the reaction gas is generally composed of hydrocarbons, such as methane, for example, or else of inert gases such as nitrogen. In addition there may also be other substances present in the reaction gas, such as steam, carbon dioxide or noble gases.

The above-described constituents of the reaction mixture may optionally each contain small amounts of impurities. Ethylene may be used, for example, in any state of purity that is suitable for the gas phase oxidation of the invention. Suitable states of purity include, but are not limited to, polymer-grade ethylene, which typically has a purity of at least 99%, and chemical-grade ethylene, which has a low purity of typically less than 95%. The impurities are typically composed primarily of ethane, propane and/or propene.

The epoxidation is carried out typically at elevated temperature. Preferred temperatures are those in the range from 150 to 350° C., more preferably in the range from 180 to 300° C., more preferably in the range from 190 to 280° C., and very preferably in the range from 200 to 280° C. The present invention accordingly also provides a process as described above wherein the oxidation takes place at a temperature in the range from 180 to 300° C., preferably in the range from 200 to 280° C. The invention likewise provides the catalyst obtainable by this process.

It is preferred to operate at pressures in the range from 5 bar to 30 mbar. With further preference the oxidation takes place at a pressure in the range from 5 bar to 25 bar, preferably at a pressure in the range from 10 bar to 20 bar, and more particularly in the range from 14 bar to 20 bar. The present invention, accordingly, also provides a process as described above wherein the oxidation takes place at a pressure in the range from 14 bar to 20 bar. The invention likewise provides the catalyst obtainable by this process.

The oxidation is carried out preferably in a continuous process. Where the reaction is carried out continuously, a GHSV (gas hourly space velocity) is used, depending on the nature of the reactor selected, as for example on the size/average area of the reactor, the shape and the size of the catalyst, that is preferably in the range from 800 to 10000/h, preferably in the range from 2000 to 6000/h, more preferably in the range from 2500 to 5000/h, the figures being based on the volume of the catalyst.

The preparation of ethylene oxide from ethylene and oxygen may be carried out advantageously in a circulation procedure. In this case the reaction mixture is passed in a circuit through the reactor, with the newly formed ethylene oxide and also the byproducts formed during the reaction being removed from the product gas stream after each traversal, and this product gas stream, following supplementation with the required amounts of ethylene, oxygen and reaction moderators, being passed back into the reactor. The removal of the ethylene oxide from the product gas stream and its processing may take place in accordance with the customary methods of the prior art (cf. Ullmann's Encyclopedia of Industrial Chemistry, 5^{th} edition, volume A-10, pp. 117-135, 123-125, VCH-Verlagsgesellschaft, Weinheim 1987).

Furthermore, accordingly, the present invention also provides for the use of a catalyst for preparing ethylene oxide, at least comprising silver, applied to a support, the support having a BET constant C in the range from 0 to 800, determined in accordance with DIN ISO 9277, or, as described above, the use of a catalyst obtainable by a process for preparing a catalyst for preparing ethylene oxide, as described above, as a catalyst for preparing ethylene oxide.

Particularly preferred embodiments of the invention are indicated below, including the embodiments resulting from the combinations given by the back-references being explicitly:
1. A catalyst for preparing ethylene oxide, at least comprising silver applied to a support, the support having a BET constant C in the range from 0 to 800, determined in accordance with DIN ISO 9277.
2. The catalyst according to embodiment 1, wherein the support is an alumina support, preferably an alpha alumina support.
3. The catalyst according to embodiment 2, wherein the alumina has a purity of at least 85%.
4. The catalyst according to any of embodiments 1 to 3, wherein the support has a bimodal pore distribution, preferably a bimodal pore distribution at least comprising pores having pore diameters in the range from 0.1 to 10 μm and pores having pore diameters in the range from 12 to 100 μm, determined by Hg porosymmetry in accordance with DIN 66133.
5. The catalyst according to any of embodiments 1 to 4, wherein the catalyst comprises silver in an amount of 5% to 35% by weight.
6. The catalyst according to any of embodiments 1 to 5, wherein the catalyst comprises rhenium, preferably in an amount of 50 to 600 ppm, based on the total weight of the catalyst and calculated as the element.
7. The catalyst according to any of embodiments 1 to 6, wherein the catalyst further comprises at least one further promoter, preferably a promoter selected from the group consisting of tungsten, lithium, sulfur, cesium, chromium, manganese, molybdenum, potassium, and mixtures of two or more thereof.
8. A catalyst support for a catalyst for preparing ethylene oxide, the support having a BET constant C in the range from 0 to 800, determined in accordance with DIN ISO 9277.
9. The support according to embodiment 8, wherein the support is an alumina support, preferably an alpha alumina support.
10. The support according to embodiment 9, wherein the alumina has a purity of at least 85%.
11. The support according to any of embodiments 8 to 10, wherein the support has a bimodal pore distribution, preferably a bimodal pore distribution at least comprising pores having pore diameters in the range from 0.1 to 10 μm and pores having pore diameters in the range from 12 to 100 μm, determined by Hg porosimetry in accordance with DIN 66133.
12. A process for preparing a catalyst for preparing ethylene oxide, at least comprising the steps of
   (i) providing a support having a BET constant C in the range from 0 to 800, determined in accordance with DIN ISO 9277;
   (ii) applying silver to the support from (i).
13. A catalyst obtainable or obtained by a process according to embodiment 12.
14. A process for preparing ethylene oxide from ethylene, comprising oxidizing ethylene in the presence of a catalyst according to any of embodiments 1 to 7 and 13.
15. The use of a catalyst according to any of embodiments 1 to 7 and 13 for preparing ethylene oxide from ethylene.

The present invention is illustrated below with reference to examples.

### EXAMPLES

### 1. Catalysts

Catalysts were produced on the basis of 5 different supports, the supports differing in their C values, determined in accordance with DIN ISO 9277:

The composition of the supports is indicated in table 1.

TABLE 1
Supports used

| Support | 1 | 2 * | 3 * | 4 * | 5 |
|---|---|---|---|---|---|
| Ring size | 6.07 × | 6.03 × | 8.00 × | 8.17 × | 6.03 × |
| [mm × mm × mm] | 5.7 × | 5.59 × | 7.55 × | 7.47 × | 5.46 × |
| | 2.06 | 2.35 | 2.79 | 3.21 | 2.47 |
| C value | 426 | 985 | −249 | −92 | 118 |
| Ca [ppm] | 400 | 800 | 600 | 600 | 600 |
| Mg [ppm] | 100 | 200 | 100 | 100 | 100 |
| Na [ppm] | 500 | 400 | 300 | 300 | 300 |
| K [ppm] | 300 | 100 | 100 | 100 | 100 |
| Si [ppm] | 500 | 800 | 700 | 700 | 600 |
| BET [m²/g] | 0.82 | 0.78 | 0.89 | 0.83 | 0.75 |

The supports marked with * are comparative examples

### 1.1 Preparation of the Silver Complex Solution

550 g of silver nitrate were added with stirring to an initial charge of 1.5 l of fully demineralized (FD) water and fully dissolved therein. The solution was heated to 40° C. here. 402.62 g of aqueous potassium hydroxide solution (47.8%) were mixed with 1.29 l of FD water. Then 216.31 g of oxalic acid were added and fully dissolved, and the solution was heated to 40° C. The potassium oxalate solution was subsequently added with the aid of a metering pump over the course of around 45 minutes (volume flow rate=about 33 ml/min) to the silver nitrate solution (40° C.). Following complete addition, the resulting solution was allowed to stand with stirring at 40° C. for a further hour. The precipitated silver oxalate was isolated by filtration and the filtercake obtained was washed with 1 l water portions until (around 10 l) it was free from potassium and from nitrate (determined by means of conductivity measurement on the wash solution; free from potassium and free from nitrate, respectively, means in the present instance a conductivity <40 μS/cm). As much as possible of the water was removed from the filtercake, and the residual moisture content of the filtercake was ascertained. 620 g of silver oxalate with a water content of 20.80% were obtained.

306 g of ethylenediamine were cooled to around 10° C. using an ice bath, and 245 g of water were added in small portions. Following the end of addition of the water, 484.7 g of the resulting silver oxalate (still wet) were added in small portions over the course of about 30 minutes. The mixture was stirred at RT overnight and the residue was subsequently centrifuged off. This left a clear solution, whose Ag content was determined by refractometry and whose density was determined using a 10 ml measuring cylinder.

The solution obtained contained 29.14% by weight of silver, calculated as the element, and had a density of 1.532 g/ml.

### 1.2 Preparation of the Solution Comprising Silver and Promoters

97.1004 g of the silver complex solution were introduced. Added to this initial charge were 1.1047 g of an aqueous solution of lithium and sulfur (2.85% by weight of lithium from lithium nitrate and 0.21% by weight of sulfur from ammonium sulfate), 1.791 g of an aqueous solution of tungsten and cesium (2% by weight of tungsten from tungstic acid and 3.5% by weight of cesium from cesium hydroxide 50% in H₂O), and 1.6492 g of an aqueous solution of rhenium (3.1% of ammonium perrhenate), and the solution was stirred for 5 minutes.

### 1.3 Application of the Solution to the Support

140.61 g of the support 1 (see table 1) were introduced into a rotary evaporator and evacuated. The reduced pressure was 20 mbar. The support was subjected to preliminary evacuation for around 10 minutes.

The solution obtained as per instructions 1.2 was applied dropwise to the support over the course of 15 minutes, after which the impregnated support was rotated around under reduced pressure for a further 15 minutes. Thereafter the support was left in the apparatus for 1 hour at room temperature under atmospheric pressure, with gentle mixing every 15 minutes.

### 1.4 Calcination of the Impregnated Support

The impregnated support was treated for 12 minutes at 283° C. with 8.3 m³ air per hour in a convection oven (HORO, type 129 ALV-SP, Fabr. No.: 53270).

### 1.5 Preparation of Crushed Catalyst Material

The catalyst rings obtained were coarsely crushed with a mortar in a porcelain dish. The comminuted material was subsequently brought to the desired particle size fraction (500-900 μm) using a sieving machine, circular sieve, and balls. Very hard rings were comminuted completely with the mortar and then sieved.

### 2. Epoxidation

The epoxidation was carried out in an experimental reactor composed of a vertical reaction tube of stainless steel, having an internal diameter of 6 mm and a length of 2200 mm. The reaction tube, which was jacketed, was heated with hot oil at a temperature T, which flowed through the jacket. With very good approximation, the temperature of the oil corresponds to the temperature in the reaction tube and hence to the reaction temperature.

For the epoxidation, the catalyst was used in the form of crushed catalyst material. The reaction tube was packed from bottom to top, to a height of 212 mm, with inert steatite beads (1.0-1.6 mm), above this to a height of 1100 mm with 38.2 g of crushed catalyst material, particle size 0.5-0.9 mm, and above this to a height of 707 mm with inert steatite beads (1.0-1.6 mm). The incoming gas entered the reactor from the top and left it again at the bottom end after having passed through the catalyst bed.

The incoming gas consisted of 35% by volume ethylene, 7% by volume oxygen, 1% by volume CO₂ (EC (ethylene chloride) moderation). At the beginning, 2.5 ppm of EC were used for the start-up. Depending on catalyst and performance, the EC concentration was raised every 24 hours up to a maximum of 4 ppm. The remainder of the incoming gas consisted of methane. The experiments were conducted under a pressure of 15 bar and with a gas hourly space velocity (GHSV) of 4750/h and also with a space-time yield of 250 kg EO/(m³(cat)×h).

The reaction temperature was regulated in accordance with the given ethylene oxide waste-gas concentration of 2.7%. In order to optimize the catalyst with regard to selectivity and conversion, between 2.2 and 4.0 ppm of ethylene chloride moderator were metered into the incoming gas.

The gas leaving the reactor was analyzed by means of online MS. The selectivity was determined from the results of the analysis.

The results are summarized in table 2 below.

TABLE 2
Tested support samples with different C values

| Cat. | | C | Selectivity | Temperature | Run time |
|---|---|---|---|---|---|
| No.: | Support | value | (%) | (° C.) | (h) |
| A | 1 | 426 | 90.4 | 242.8 | 200 |
| B * | 2 * | 985 | 87.4 | 238.3 | 200 |
| C * | 3 * | −249 | 87.3 | 238.9 | 200 |
| D * | 4 * | −92 | 87.8 | 240.6 | 200 |
| E | 5 | 118 | 89.2 | 238.9 | 200 |

The catalysts marked with * are comparative examples.

1. A catalyst for preparing ethylene oxide, at least comprising silver applied to a support, the support having a BET constant C in the range from 0 to 800, determined in accordance with DIN ISO 9277.

2. The catalyst according to claim 1, wherein the support is an alumina support.

3. The catalyst according to claim 2, wherein the alumina has a purity of at least 85%.

4. The catalyst according to claim 1, wherein the support has a bimodal pore distribution.

5. The catalyst according to claim 1, wherein the catalyst comprises silver in an amount of 5% to 35% by weight.

6. The catalyst according to claim 1, wherein the catalyst comprises rhenium.

7. The catalyst according to claim 1, wherein the catalyst further comprises at least one further promoter.

8. A catalyst support for a catalyst for preparing ethylene oxide, the support having a BET constant C in the range from 0 to 800, determined in accordance with DIN ISO 9277.

9. The support according to claim 8, wherein the support is an alumina support.

10. The support according to claim 9, wherein the alumina has a purity of at least 85%.

11. The support according to claim 8, wherein the support has a bimodal pore distribution.

12. A process for preparing a catalyst for preparing ethylene oxide, at least comprising the steps of
(i) providing a support having a BET constant C in the range from 0 to 800, determined in accordance with DIN ISO 9277;
(ii) applying silver to the support from (i).

13. A catalyst obtainable or obtained by a process according to claim 12.

14. A process for preparing ethylene oxide from ethylene, comprising oxidizing ethylene in the presence of a catalyst according to claim 1.

15. (canceled)

16. The catalyst according to claim 1, wherein the support is an alpha-alumina support.

17. The catalyst according to claim 1, wherein the support has a bimodal pore distribution at least comprising pores having pore diameters in the range from 0.1 to 10 μm and pores having pore diameters in the range from 12 to 100 μm, determined by Hg porosimetry in accordance with DIN 66133.

18. The catalyst according to claim 1, wherein the catalyst comprises rhenium in an amount of 50 to 600 ppm, based on the total weight of the catalyst and calculated as the element.

19. The catalyst according to claim 1, wherein the catalyst further comprises at least one further promoter wherein the promoter is selected from the group consisting of tungsten, lithium, sulfur, cesium, chromium, manganese, molybdenum, potassium, and mixtures of two or more thereof.

20. The support according to claim 8, wherein the support is an alpha-alumina support.

21. The support according to claim 8, wherein the support has a bimodal pore distribution, at least comprising pores having pore diameters in the range from 0.1 to 10 μm and pores having pore diameters in the range from 12 to 100 μm, determined by Hg porosimetry in accordance with DIN 66133.
